(19) United States
(12) Patent Application Publication
(10) Pub. No.: US 2008/0097303 A1
(43) Pub. Date: Apr. 24, 2008

(54) Syringe with retractable needle

(76) Inventors: Chih-Hsiung Chen, Taipei (TW); Kuo-Li Tseng, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/445,250

(22) Filed: Jun. 2, 2006

(51) Int. Cl. A61M 5/50

(52) U.S. Cl. 604/110

(57) ABSTRACT

A syringe with retractable needle includes a hub, a barrel, and a plunger. The hub is provided with symmetrical retaining sections, and a hooking section of a predetermined length is formed at a front end of the plunger with a piston mounted the hooking section. The piston includes a diametrically larger front part and a diametrically smaller and deformable rear part. A top surface of the piston is tightly abutted on a large lower surface of the hooking section to prevent any bubble from existing behind the hooking section. When the plunger is fully pushed forward to compress the smaller rear part of the piston, the hooking section is brought to engage with the retaining sections on the hub, so that medical liquid is fully released from the barrel, and the hub and the needle thereof can be drawn into the barrel after the injection by pulling the plunger backward.

### FIELD OF THE INVENTION

The present invention relates to a syringe with retractable needle, and more particularly to a syringe with retractable needle that effectively prevents the used needle from being used for a second time and dangerously stabbing a third party, and prevents the formation of bubbles in the syringe to ensure complete release of medical liquid for injection.

### BACKGROUND OF THE INVENTION

In addition to many syringes having a traditional structure, there are also syringes with retractable needle available in the market. The syringe with retractable needle is designed to prevent a used needle from being used for a second time and dangerously stabbing a third party. FIG. 1 is an exploded perspective view of a conventional syringe with retractable needle. As shown, the syringe includes a hub 11 having a needle 111 associated therewith, a barrel 12, and a plunger 13. The hub 11 is provided at predetermined points with retaining sections 112, and the plunger 13 is provided near a front end with a conical hooking section 131 corresponding to the retaining sections 112. A piston 14 is provided on the plunger 13 closely behind the hooking section 131. When an injection is completed, the hooking section 131 is engaged with the retaining sections 112 of the hub 11, allowing a user to pull the plunger 13 backward and draw the hub 11 and the needle 111 associated therewith into the barrel 12 to prevent the used needle 111 from being used for a second time and/or dangerously stabbing a third party. However, in the above-structured conventional syringe with retractable needle, when the hooking section 131 and the retaining sections 112 are engaged with one another, there is still a small clearance existed between the piston 14 and the hub 11. When the plunger 13 is pulled backward to draw a medical liquid into the barrel 12, particularly when the barrel 12 has a small bore for containing an amount of medical liquid less than 1 cc, there are always bubbles attached to or remained at a large rear side of the conical hooking section 131 due to a capillary action. Such bubbles are difficult to remove, and air in the bubbles tends to be injected into a patient's body to cause danger.

### SUMMARY OF THE INVENTION

A primary object of the present invention is to provide an improved syringe with retractable needle, such that the medical liquid in the syringe is free from any bubble and is completely injected into the patient's body. To achieve the above and other objects, the syringe according to the present invention includes a hub, a barrel, and a plunger. The hub is provided at predetermined positions with symmetrical retaining sections, and a hooking section is forward extended from a front end of the plunger by a predetermined length. Wherein, a piston is mounted around the hooking section. The piston is a stepped piston having a front part forming a hollow cylinder with a larger outer diameter, and a rear part forming a hollow cylinder with a smaller outer diameter. The piston is provided with a central fixing bore to fitly bear on the hooking section, such that a top surface of the piston is tightly abutted on a large rear surface of the hooking section without any clearance left between the piston and the hooking section. When the plunger received in the barrel is pulled backward to draw a medical liquid into the barrel before an injection, since there is not any clearance left between the piston and the hooking section, no bubble will occur between the piston and the hooking section. When the plunger is fully pushed forward to inject, the hooking section is brought to engage with the retaining sections on the hub, allowing the hub and the needle thereof to be drawn into the barrel after the injection by pulling the plunger backward. Moreover, when the plunger is fully pushed forward to complete the injection, the diametrically smaller rear part of the piston is compressed to deform, producing a second time of pushing and compressing against the medical liquid being injected, so that the medical liquid is fully released from the barrel of the syringe.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is an exploded perspective view of a conventional syringe with retractable needle;

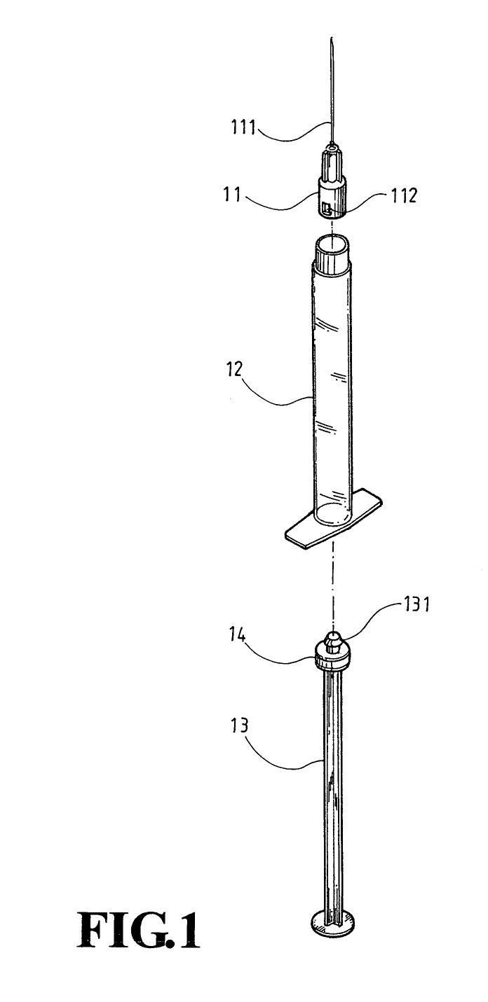

FIG. 2 is an assembled perspective view of a syringe with retractable needle according to a preferred embodiment of the present invention;

FIG. 3 is an exploded view of FIG. 2; and

FIGS. 4 and 5 are fragmentary sectioned side views showing the operation of the syringe with retractable needle according to the present invention.

### DETAILED DESCRIPTION OF THE PREFERRED EMBODIMENTS

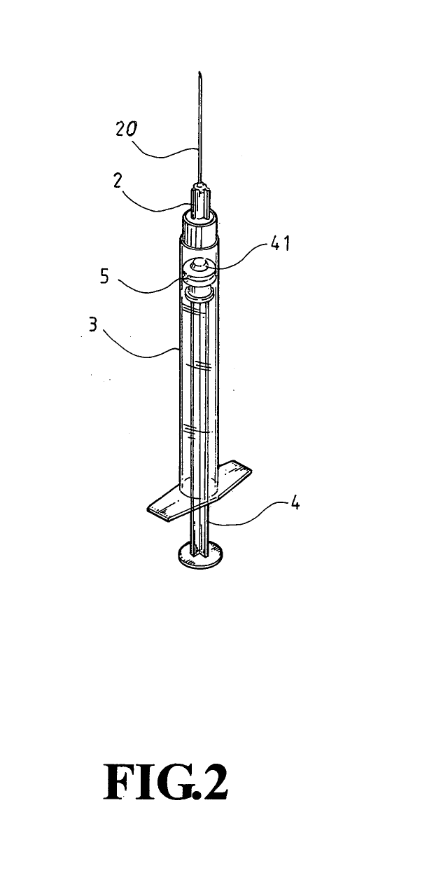

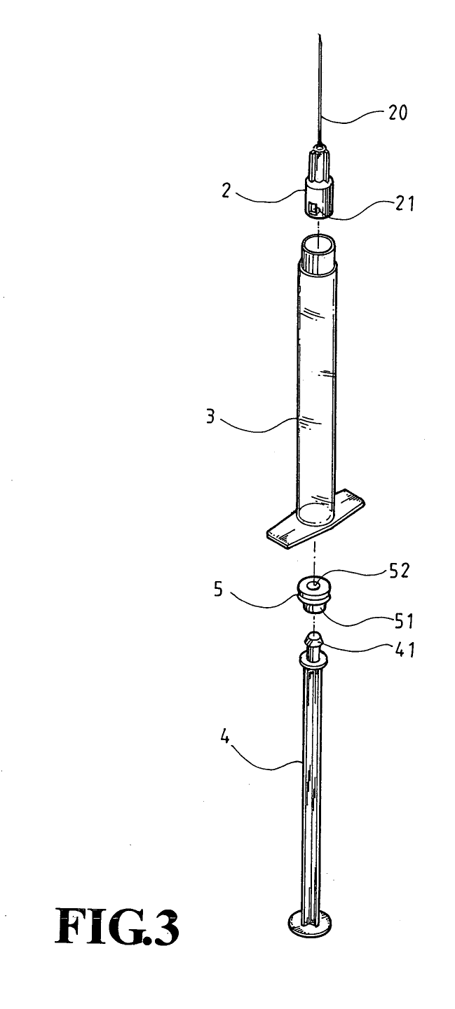

Please refer to FIGS. 2 and 3 that are assembled and exploded perspective views, respectively, of a syringe with retractable needle according to a preferred embodiment of the present invention. As shown, the syringe of the present invention includes a hub 2 having a needle 20 associated therewith, a barrel 3, and a plunger 4. The hub 2 is provided at predetermined positions with symmetrical retaining sections 21. The hub 2 is tightly fitted in a front opening of the barrel 3 with a predetermined binding force. The plunger 4 is provided at a front end with a hooking section 41 of a predetermined length. A piston 5 is mounted around the hooking section 41.

The piston 5 is a stepped piston defining two different outer diameters. A front part of the piston 5 is a hollow cylinder having a larger outer diameter, while a rear part 51 of the piston 5 is a hollow cylinder having a smaller outer diameter rearward extended from the front part by a predetermined distance. The piston 5 has a central fixing bore 52 adapted to tightly bear on the hooking section 41. Due to the stepped diameter design, the diametrically larger front part of the piston 5 has hardness larger than that of the diametrically smaller rear part of the piston 5. With the increased hardness, the larger front part of the piston 5 is not subject to deformation and able to keep an airtight contact with the barrel 3 and the hub 2 when the plunger 4 is pushed forward in the injection. Moreover, the piston 5 is mounted around the hooking section 41 with a top surface of the piston 5 tightly bearing on a large rear surface of the hooking section 41 without any clearance left between the piston 5 and the hooking section 41.

When the plunger 4 is fully received in the barrel 3, the top surface of the piston 5 is fitly pressed against an inner end wall of the hub 2. When the plunger 4 in this position is pulled backward to draw a medical liquid into the barrel 3 via the needle 20 and the hub 2 for injection, since the top surface of the piston 5 is in tight contact with the rear surface of the hooking section 41 without any clearance left between them, no bubble would come into and exist between the hooking section 41 and the piston 5. When the plunger 4 is fully pushed forward to inject, the hooking section 41 is brought to engage with the retaining sections 21 on the hub 2, allowing the hub 2 and the needle 20 thereof to be drawn into the barrel 3 after the injection by pulling the plunger 4 backward. Therefore, the used needle 20 is prevented from being used for a second time and/or dangerously stabbing a third party. Moreover, when the plunger 4 is fully pushed forward to complete the injection, the diametrically smaller rear part 51 of the piston 5 is compressed to deform, producing a second time of pushing and compressing against the medical liquid being injected, so that the medical liquid is fully released from the barrel 3 of the syringe. Meanwhile, the diametrically larger front part of the piston 5, due to the increased hardness thereof, keeps in airtight contact with the inner end wall of the hub 2 to ensure that all the medical liquid is released from the barrel 3.

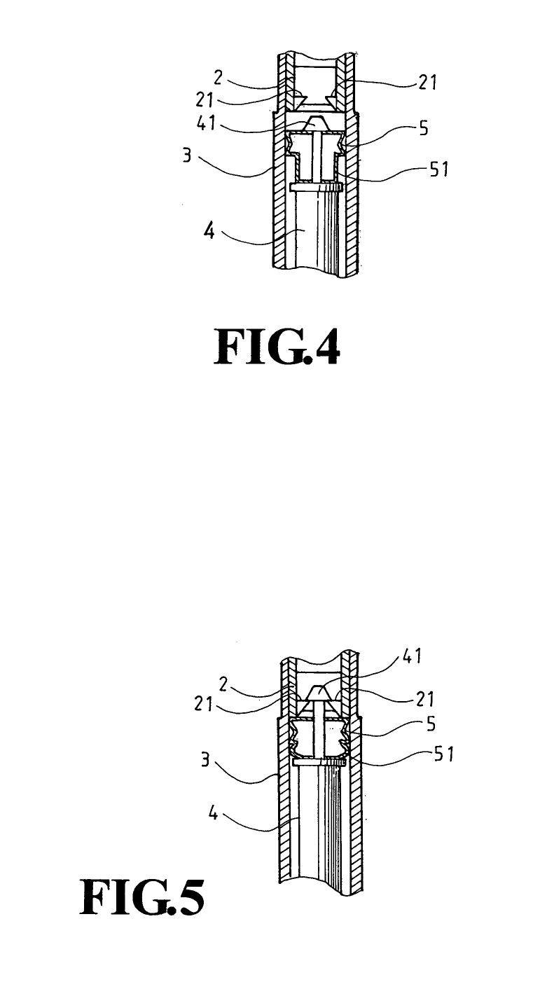

Please refer to FIGS. 4 and 5 at the same time, which show the operation of the syringe of the present invention. To inject, the plunger 4 is fully pushed forward, so that the medical liquid in the barrel 3 is pushed forward smoothly. When the plunger 4 is almost fully pushed into the barrel 3 at a final stage of injection, the piston 5 at the front end of the plunger 4 would finally press against the hub 2 fitted in the front opening of the barrel 3 while the hooking section 41 of the plunger 4 can still be pushed forward to pass through the retaining sections 21 in the hub 2 and be held thereto. When the hooking section 41 is kept pushing forward while the piston 5 has been stopped from moving any further by the hub 2, the diametrically smaller rear part 51 of the piston 5 is compressed to produce a second time push against the medical liquid until the hooking section 41 is engaged with the retaining sections 21 of the hub 2 and the medical liquid is completely released from the barrel 3.

It is noted the top surface of the piston 5 is in tight contact with the rear surface of the hooking section 41. Therefore, unlike the conventional syringes with retractable needle, no bubble will occur in the present invention to cause unreleased medical liquid in the barrel 3 when the plunger 4 is engaged with the hub 2.

What is claimed is:

1. A syringe with retractable needle, comprising a hub having a needle associated therewith, a barrel for containing a medical liquid therein, and a plunger slidably received in said barrel; said hub being tightly fitted in a front opening of said barrel and provide at predetermined positions with symmetrical retaining sections, and said plunger being provided at a front end with a hooking section of a predetermined length; said syringe being characterized in that a piston is further provided around said hooking section; said piston including a diametrically larger front part in the form of a hollow cylinder, and a diametrically smaller rear part in the form of a hollow cylinder to rearward extend from said front part by a predetermined distance; a top surface of said piston being fitly contacted with a large rear surface of said hooking section to prevent formation of any bubble between said piston and said hooking section; whereby when said plunger is pushed forward in said barrel with said stopped from moving any further by said hub, said diametrically smaller rear part of said piston can be compressed and deformed to allow said plunger to move further to produce a second time push against the medical liquid in said barrel and cause said hooking section to pass through and engage with said retaining sections on said hub, allowing the medical liquid to be completely released from said barrel and said hub and said needle to be drawn into said barrel after injection by pulling said plunger backward.
